(19) United States
(12) Patent Application Publication
(10) Pub. No.: US 2024/0247320 A1
(43) Pub. Date: Jul. 25, 2024

(54) METHODS FOR IDENTIFYING CRITICALLY SHORT TELOMERES

(71) Applicant: Centarix Biotech Ltd, Jerusalem (IL)

(72) Inventors: Noam Maoz, Jerusalem (IL); Yitzchak Dershowitz, Jerusalem (IL); Shimon Meshi, Jerusalem (IL); Hodaya LAUFER, Jerusalem (IL)

(73) Assignee: Centarix Biotech Ltd, Jerusalem (IL)

(21) Appl. No.: 18/564,090

(22) PCT Filed: May 26, 2022
(86) PCT No.: PCT/IL2022/050558

§ 371 Date: Nov. 26, 2023

Related U.S. Application Data
(60) Provisional application No. 63/193,165, filed on May 26, 2021.

(51) Int. Cl. C12Q 1/6886; C12Q 1/6806

(52) U.S. Cl. CPC C12Q 1/6886; C12Q 1/6806; C12Q 2600/156

(57) ABSTRACT

Described herein are high throughput-adaptable methods of detecting and quantifying telomere sequences, and particularly telomere sequences of shorter than average length, starting from extracted genomic DNA. Applications of the noted methods, such as to characterize age and health of a subject, for screening active agents to increase cellular longevity, and assessing the efficacy of treatments for diseases associated with shorter than average telomeres are also described.

### CROSS-REFERENCE TO RELATED APPLICATIONS

Benefit is claimed to U.S. Provisional Patent Application No. 63/193,165, filed on May 26, 2021, the contents of which are incorporated by reference herein in their entirety.

### FIELD

This disclosure relates to methods of detecting and quantifying telomere sequences, and particularly telomere sequences of shorter than average length in a subject over time. Applications of the noted methods, such as to characterize age and health of a subject, for screening active agents to increase cellular longevity, and assessing the efficacy over time of treatments for diseases associated with shorter than average telomeres are also described.

### BACKGROUND

Telomeres are found at the ends of linear chromosomes across the spectrum of vertebrate, invertebrate, plant, and microorganisms. Telomeres function to solve the “lagging strand problem” of DNA replication, and provide a nucleic acid and nucleoprotein buffer between progressively shortening chromosome ends and structural genetic sequence located proximal to chromosomal termini. Average telomere length varies between telomere possessing species; however, it is well-recognized that average telomere length within a species decreases with chronological age. Moreover, it is now recognized that shorter than average telomere length, and particularly the presence of critically short telomere length in a telomere possessing species can be a hallmark of biological disfunction at the cellular, organ, or even organismal level.

Multiple methods of measuring telomere length were previously developed (see Lai et al, Phil. Trans. R. Soc. B373 20160451, the contents of which are incorporated by reference herein with respect to the telomere detection methods described therein). Quantitative PCR (qPCR)-mediated methods have also been described for use with digital drop PCR methodologies (see U.S. Pat. No. 9,347,094). However, none of the described methods allow for high throughput detection and relative quantification of critically short telomeres, which do not require intact cells. The ability to measure critically short telomeres from extracted genomic DNA and not by using intact cells is important in many clinical scenarios, in particular to detect the genomic response to therapies for diseases and conditions known to be evidenced by the presence of critically short telomeres. Thus, a continuing need exists for methods that can easily identify critically short telomeres in a high throughput manner, from genomic DNA extracts.

### SUMMARY

Described herein is a high throughput-adaptable method for quantifying critically short telomeres, starting from a DNA sample, in a subject from a telomere-possessing species or sample therefrom, which includes the steps of: digesting DNA from a subject or sample therefrom with at least one restriction enzyme, wherein the at least one restriction enzyme cuts proximal to, but does not cut within, a telomeric sequence; separating the digested DNA by size; isolating from the separated DNA, polynucleotide fragments shorter in length than an average telomere length for the sample; amplifying from the isolated DNA: (a) a telomeric-specific sequence; and (b) a non-telomeric single copy non-telomeric sequence; quantitating the products of (a) and (b); and determining an amount of the critically short telomeres by dividing the quantitated amount of (a) by the quantitated mount of (b).

In particular embodiments, the DNA is digested with multiple restriction enzymes.

In other particular embodiments, the digested DNA is separated by gel electrophoresis.

In still other particular embodiments, the polynucleotide fragments are isolated from the gel, and can then, in certain embodiments, be isolated from the gel by removing a gel section containing the fragments, and eluting the DNA from the gel section.

In some embodiments, polynucleotide fragments no larger than 3.0 or 1.5 kilobase in length are isolated, such as polynucleotide fragments between about 0.5 kilobase and about 1.5 kilobase in length, between about 1.0 kilobase and about 1.5 kilobase in length, or between about 1.5 kilobase and about 3.0 kilobase in length.

In other particular embodiments of the described method, amplification reactions (a) and (b) are amplified in the same PCR reaction.

In still other particular embodiments, amplification and quantitation of amplification reactions (a) and (b) are by quantitative PCR, such as real time PCR or digital PCR, including digital droplet PCR.

In other particular embodiments, the subject is a human subject.

In still other embodiments, the sample is a blood or buccal sample.

Further described herein are methods of diagnosis, prognosis, and treatment of diseases and conditions related to relative increases in cs telomeres in a subject. Similarly described herein are methods that use the described cs telomere quantitation methods as part of high throughput screening methods to identify agents, such as small molecule compounds and biomolecules (e.g., nucleic acids and proteins), that can influence the relative amount of cs telomeres in a cell, thereby treating the disease or condition.

Lastly, kits for carrying out the noted methods are also described.

The foregoing and other objects, features, and advantages will become more apparent from the following detailed description, which proceeds with reference to the accompanying figures.

### BRIEF DESCRIPTION OF THE DRAWINGS

FIG. 1 is a graph showing linear behavior observed for addition of critically short telomere fragments. Different amounts of a telomere fragment of the size 900 bp were added to a human genomic DNA sample, and then critically short telomere amount was measured using the protocol developed herein. Telomeric sequences and the single copy non-telomeric control were amplified and detected in a gel fragment between 0.5 and 1.5 kb.

FIG. 2 is a graph showing measurement critically short telomeres from a biologically older adult that were added in defined relative amounts (X-axis) to a genomic sample from a biologically younger adult. Telomeric sequences and the single copy non-telomeric control were amplified and detected in a gel fragment between 1.5 and 3.0 kb.

FIGS. 3A and 3B are graphs demonstrating telomere length measurements from whole blood samples from 13 people of ages 19 to 89 years. (FIG. 3A) Critically short telomeres were measured using the method described herein. (FIG. 3B) Average telomere length for intact DNA was measured by ddPCR using the same primers and probes as in (FIG. 3A). Telomeric sequences and the single copy non-telomeric control were amplified and detected in a gel fragment between 0.5 and 1.5 kb.

FIG. 4 is a graph showing that a 1:1 Mix of DNA samples from two people with different levels of critically short telomeres produce the expected average. Telomeric sequences and the single copy non-telomeric control were amplified and detected in a gel fragment between 0.5 and 1.5 kb.

FIGS. 5A and 5B are graphs showing measurement of critically short telomere in cultured fibroblasts after 6 and 51 passages (p.6 and p.51, respectively). FIG. 5A shows the results using the methods described herein. FIG. 5B shows the results using the TeSLA blot method, as described. Telomeric sequences and the single copy non-telomeric control were amplified and detected in a gel fragment between 1.5 and 3.0 kb.

### BRIEF DESCRIPTION OF THE DESCRIBED SEQUENCES

The nucleic sequences provided herewith are shown using standard letter abbreviations for nucleotide bases, as defined in 37 C.F.R. 1.822. Only one strand of each nucleic acid sequence is shown, but where applicable, the complementary strand is understood as included by any reference to the displayed strand. The Sequence Listing is submitted as an ASCII text file named 3231_1_2_SeqList.txt, created May 25, 2022, about 2 KB, which is incorporated by reference herein.

SEQ ID Nos 1 and 2 are forward and reverse telomere-specific PCR primers.

SEQ ID Nos 3 and 4 are forward and reverse PCR primers for amplifying a single-copy non-telomeric sequence.

SEQ ID NO: 5 is an oligonucleotide sequence for probing the presence of an amplified telomere sequence.

SEQ ID Nos 6 and 7 are forward and reverse PCR primers for amplifying a single-copy genomic sequence.

SEQ ID NO: 8 is an oligonucleotide sequence for probing the presence of an amplified single-copy genomic sequence.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.,” is derived from the Latin exempli gratia, and is used herein to indicate a non-limiting example. Thus, the abbreviation “e.g.,” is synonymous with the term “for example.”

In case of conflict, the present specification, including explanations of terms, will control. In addition, all the materials, methods, and examples are illustrative and not intended to be limiting.

Abnormal: Deviation from normal characteristics. Normal characteristics can be found in a control, including a standard for a population, such as the average telomere length for a subject of a particular age in a telomere possessing species. For instance, where the abnormal condition is a disease condition, a few appropriate sources of normal characteristics might include an individual who is not suffering from the disease, or a population standard of individuals believed not to be suffering from the disease. In particular embodiments, an abnormally long or short telomere length (e.g., longer or shorter than normal) can signify that a subject has a disease or condition or is predisposed to the disease or condition even if no symptoms are apparent. The efficacy of treatment, even prophylactic treatment, can be measured by detecting the presence, lessening, or absence of an abnormal characteristic over the course of the treatment.

Amplification: When used in reference to a nucleic acid, any technique that increases the number of copies of a nucleic acid molecule in a sample or specimen. An example of amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification can be characterized by electrophoresis, restriction endonuclease cleavage patterns, oligonucleotide hybridization or ligation, and/or nucleic acid sequencing, using standard techniques. Other non-limiting examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Animal: Living multi-cellular vertebrate organisms, a category that includes for example, mammals and birds. The term mammal includes both human and non-human mammals. Similarly, the term subject includes both human and veterinary subjects, for example, humans, non-human primates, dogs, cats, horses, cows, pigs, and rodents. More broadly, the term “subject” includes human, veterinary, and even non-animal telomere-possessing species, which encompasses any organism that possesses telomeres. Non-limiting general examples of telomere possessing species include animals, insects, and fungi such as yeast.

Biological age: Health status of an organism or cell, as can be determined by relevant biomarkers. Correlates with the expected remaining lifespan of an organism, or division potential for mitotic cells in tissue culture. Biological age correlates with chronological age, but organisms with the same chronological age can have different biological age.

Biological Sample: Any sample that may be obtained directly or indirectly from an organism, including blood and buccal samples from a mammalian subject. A biological sample for use in the described methods is any sample that includes telomere-containing nucleic acids. In a particular embodiment, a biological sample is any amount of cellular material containing genomic DNA that can be processed and amplified as described herein. In other embodiments, the biological sample is telomere-containing DNA that has been isolated from a subject or sample therefrom. Biological samples for use in the described methods are collected or obtained using methods well known to those skilled in the art.

Cancer: A malignant disease characterized by the abnormal growth and differentiation of cells. The DNA of such cells often maintain long telomere sequence length beyond normal chronological age of the cancer cells, i.e., beyond what would be expected for a cell of the given chronological age. “Metastatic disease” refers to cancer cells that have left the original tumor site and migrate to other parts of the body for example via the bloodstream or lymph system. “Gynecological cancers” include cancers of the uterus (for example endometrial carcinoma), cervix (for example cervical carcinoma, pre-tumor cervical dysplasia), ovaries (for example ovarian carcinoma, serous cystadenocarcinoma, mucinous cystadenocarcinoma, endometrioid tumors, celioblastoma, clear cell carcinoma, unclassified carcinoma, granulosa-thecal cell tumors, Sertoli-Leydig cell tumors, dysgerminoma, malignant teratoma), vulva (for example squamous cell carcinoma, intraepithelial carcinoma, adenocarcinoma, fibrosarcoma, melanoma), vagina (for example clear cell carcinoma, squamous cell carcinoma, botryoid sarcoma), embryonal rhabdomyosarcoma, and fallopian tubes (for example carcinoma). “Breast cancer” includes cancers of the breast tissue, such as adenocarcinoma. The most common type of breast cancer is ductal carcinoma. Ductal carcinoma in situ is a non-invasive neoplastic condition of the ducts. Lobular carcinoma is not an invasive disease, but it is an indicator that a carcinoma may develop.

Chronological age: The age of an organism as a function of time, for example weeks or years.

Contacting: Placement in direct physical association. Includes both in solid and liquid form. Contacting can occur in vitro with isolated cells or in vivo by administering to a subject. The effect of a test agent on a given cell or tissue is determined by contacting the cell or tissue with the test agent.

Control: A reference standard. A control can be a known value indicative of average telomere length of a healthy subject. Another example of a control in the context of the described methods is the average value of telomere length for cells of a known chronological or cellular age, for example cells that have divided a known number of times. In particular examples a control sample is taken from a subject that is known not to have a disease or condition. In other examples a control is taken from the subject being diagnosed, but at an earlier time point, either before disease onset or prior to or at an earlier time point in disease treatment. In still other embodiments a control sample can be from a subject of any age that is determined to be the reference age for a given comparison.

A difference between a test sample and a control can be an increase or conversely a decrease. The difference can be a qualitative difference or a quantitative difference, for example a statistically significant difference. In some examples, a difference is an increase or decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Critically short telomeres (critically shortened telomeres): In the absence of functional telomerase, or as a result of natural ageing, or certain cellular pathologies, telomeres become progressively shorter with each successive round of DNA replication and cell division. Telomeres that are critically short are more likely to be recognized by the cell as having an unrepairable double stranded DNA break which can be a triggering factor for cellular senescence or apoptosis, and moreover can result in aneuploidy following chromosomal end joining. As an organism ages, or in the context of certain diseases and conditions, the proportion of critically short telomeres is increased in comparison to the same organism of younger age or in the absence of the disease or condition. Telomeres of varying species are recognized as critically short at varying lengths, but share the common characteristic of being shorter than average telomere length of a given telomere possessing species. In humans, critically short telomeres have been defined as being of varying lengths, with the greatest length being defined as below about 3.0 kb (Quintela-Fandino et al., Oncotarget 8:21472-21482, 2017).

Critically Short Telomere score (csTelomere score): The relative amount of critically short telomeres within the cells of an organism. csTelomere score can affect biological age. Detect: To determine if an agent (such as a signal or particular nucleic acid) is present or absent, often through use of a detectable label. In some examples, this can further include quantification.

Diagnosis: The process of identifying a disease or a predisposition to developing a disease by its signs, symptoms, and results of various tests and methods, for example the methods disclosed herein. The conclusion reached through that process is also called “a diagnosis.” Forms of testing commonly performed include blood tests, medical imaging, urinalysis, PAP smear, and biopsy. While such tests can be used independently of each other, they are not mutually exclusive when forming a diagnosis. For example, detection and quantification of critically short telomeres can be used to complement another diagnostic test. The term “predisposition” refers to an effect of a factor or factors, such as a greater proportion than normal of critically-short telomeres, that render a subject susceptible to a condition, disease, or disorder, such as cancer. In some examples, of the disclosed methods, testing is able to identify a subject predisposed to developing a condition, disease, or disorder, associated with increased csTelomere score. In particular embodiments, a given treatment for a disease or condition is provided as the direct result of a particular diagnosis, in other embodiments, a diagnostic test is given at multiple time points to determine progression of a disease or condition, and/or the response thereof to a given treatment, over time.

Effective amount of a compound: A quantity of compound sufficient to achieve a desired effect in a subject being treated. An effective amount of a compound can be administered in a single dose, or in several doses, for example daily, during a course of treatment. However, the effective amount of the compound will be dependent on the compound applied, the subject being treated, the severity and type of the affliction, and the manner of administration of the compound. In particular embodiments, the effective amount of a compound is the amount necessary in vitro to affect a parameter, such as proportion of critically short telomeres in a sample.

Increased risk: As used herein “increased risk” of a condition refers to an increase in the statistical probability of developing a condition, such as a cancer, relative to the general population. A subject that is predisposed to a condition is also said to be at increased risk of the condition.

Interstitial telomeric sequences (ITSs): Consist of tandem repeats of the canonical telomeric repeat and are common in mammals. ITSs are localized at intrachromosomal sites, for example close to centromeres, as well as at interstitial sites, between the centromeres and the telomeres. ITS amounts vary between individuals; however, it is assumed that ITSs are typically below 500 bp in length, but can be of greater length.

Isolated: A biological component (such as a nucleic acid molecule, protein or organelle) that has been substantially separated or purified away from other biological components in the cell of the organism in which the component naturally occurs, i.e., other chromosomal and extra-chromosomal DNA and RNA, proteins and organelles. Nucleic acids and proteins that have been isolated include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule to facilitate detection of that molecule. Specific, non-limiting examples of labels include radioactive isotopes, enzyme substrates, co-factors, ligands, chemiluminescent or fluorescent agents, haptens, and enzymes. In particular embodiments for use with the methods described herein, a detectable fluorescent label is conjugated to a specific nucleic acid probe for use in ddPCR amplification to detect and/or quantify a PCR amplification product. In specific embodiments, a label is conjugated to a probe for detecting an amplified telomeric sequence and a different label is conjugated to a probe for detecting an amplified single copy gene sequence.

Mitotic age: The age of cells in culture as measured by number of cell divisions. The biological age of cells in culture correlates with the number of passages a culture undergoes.

Purified: The term purified does not require absolute purity; rather, it is intended as a relative term. Thus, for example, a purified protein preparation is one in which the protein referred to is purer than the protein in its natural environment within a cell.

Quantitative PCR (qPCR): A method for detecting and measuring products generated during each cycle of a PCR, which products are proportionate to the amount of template nucleic acid present prior to the start of PCR. The information obtained, such as an amplification curve, can be used to quantitate the initial amounts of template nucleic acid sequence. Non-limiting examples of qPCR include quantitative real time PCR, digital droplet PCR (ddPCR), and the like.

Restriction enzyme: Restriction enzymes (or restriction endonucleases) are proteins that cut DNA at specific sequences. Hundreds of restriction enzymes exist, both isolated from natural sources (e.g., from bacteria and archaea) and synthetically modified for use under certain temperature and reaction conditions, and which each recognize and cut DNA at an enzyme-specific sequence. DNA cut with a restriction enzyme is also described as having been “digested”. The resultant DNA products of a restriction digest can be referred to as restriction fragments, the size of which will depend on the location of restriction recognition and cut sites and the enzymes used in a given digest.

Screening: As used herein, “screening” refers to the process used to evaluate and identify candidate agents that exhibit a desired function or activity, for example an agent that slows the development of critically short telomeres.

Senescence: Cellular senescence is defined as a condition in which a cell no longer has the ability to proliferate. Senescent cells are irreversibly arrested at the G0 or G1 phase of the cell cycle and do not respond to various external stimuli, but they remain metabolically active. One biological hallmark of a senescent cell is greater than average proportion of critically short telomeres.

Survival (of a cell): The length of time a given cell is alive. An increase in survival following treatment indicates that the cell lives for a longer length of time as compared to a control, such as the cell in the absence of treatment.

Target sequence: A target sequence is a portion of DNA to which an oligonucleotide primer hybridizes, for example in an amplification reaction. Also embraces a DNA sequence targeted for amplification by the methods described herein (“amplification target sequence”), such as a telomeric sequence or single-copy non-telomeric sequence found within a restriction enzyme generated, and telomere-containing DNA fragment Telomerase: The enzyme (DNA polymerase) primarily responsible for repairing damage to the structures at the end of linear chromosomes known as telomeres. Telomerase adds specific DNA sequence repeats (TTAGGG in all vertebrates) to the 3′ end of DNA strands in telomeres, which are found at the ends of eukaryotic chromosomes. Telomerase functions as a reverse transcriptase, and is associated with an RNA molecule that acts as a template for elongating telomeres that have been shortened after replication.

Test compound: A compound used in a test or screen, and which can be essentially any compound, such as a small molecule, a chemotherapeutic, a polypeptide, a hormone, a nucleic acid, a modified nucleic acid, a sugar, a lipid and the like. Test compounds are used, for example, when screening for compounds that block the telomere shortening, or conversely activate telomerase activity.

### II. Methods for Detecting and Quantifying Critically Short Telomeres

An increased proportion of critically short telomeres is a natural hallmark of chronologically older organisms, and is associated with a range of diseases and conditions. Although several methods exist to detect telomeres, most do not provide resolution sufficient to detect what may overall be a relatively small but biologically significant increased proportion of critically short telomeres, while those methods that can detect critically short telomeres (TESLA, HT Q-FISH), are either not adaptable for high-throughput use or cannot be performed on genomic DNA extracts, both important requirements in many clinical settings. Additionally, HT Q-FISH cannot differentiate between telomeres of 3 kb and those shorter than 3 kb. Moreover, because HT Q-FISH takes an image of the nucleus to measure the telomeres while cells are at the interphase stage of the cell cycle, there is significant telomere clustering which interferes with telomere measurement. The present disclosure relates to methods that overcome these problems of detecting critically short telomere “signal” amidst average length telomere “noise”, in a high throughput manner, from genomic DNA extracts. Particular embodiments of the described methods include measurement of this signal in a given subject over time; such that despite the presence of any interstitial telomeric sequences (ITSs), the presence and increased or decreased abundance of critically short telomeres can clearly be detected.

Described herein are methods for detecting and quantifying critically short telomeres. In a first step, sample DNA is digested by at least one restriction enzyme, wherein the at least one restriction enzyme does not cut within telomeric sequence. The resultant restriction fragments are then separated by size, and restriction fragments are isolated that are shorter in length than an average telomere length for the sample from which the DNA is taken. From the isolated DNA, at least two target sequences are amplified: (a) a telomeric-specific sequence; and (b) a single copy non-telomeric sequence that serves as a non-telomeric loading control. The relative amount of critically short telomeres is determined by dividing the quantitated amount of (a) by the quantitated mount of (b). In particular embodiments, this method is repeated on a sample taken from the same subject at a later time point. In other particular embodiments, the method is used to compare the presence of critically short telomeres in samples taken from the same subject at two or more time points, such as three, four, five, six, seven, eight, or even more time points. In particular embodiments, the samples are taken hours, days, weeks, months, or even years apart.

The methods described herein detect and quantify critically short telomeres in a DNA sample isolated from a telomere possessing species. Telomere length varies by species and even by chromosome within a species. By extension, the length of a telomere that is deemed critically short also varies. The methods described herein are adaptable to these noted variations. In particular embodiments, a critically short telomere can be less than 500 nucleotides in length, less than 1 kilobase (kb), less than 1.5 kb, less than 2 kb, less than 2.5 kb, less than 3 kb, less than 3.5 kb, less than 4.5, less than 5 kb, or even more, depending on the species and the knowledge of the art with respect to classification of critically short telomeres for the particular species. The described methods can also detect and quantify critically short telomeres that are 100, 200, 300, 400, or 500 nucleotides in length. In other embodiments, such as but not limited to detecting and quantifying critically short telomeres in a human subject, the described methods detect and quantify critically short telomeres in genomic fragments that are 100 nt-5 kb long, and all increments in between, such as 0.1-1.0 kb, 1.0-2.0 kb, 0.5-1.5 kb, 1.5-3.0 kb, and the like such as up to about 3.0 kb in length or even up to about 5.0 kb in length.

The described methods detect and quantify critically short telomeres in a subject from a telomere possessing species, or in a DNA-containing sample taken from a telomere possessing species. Such species include any organism that has linear chromosomes and therefore require telomeres to buffer against the shortening of chromosomal ends that naturally result from the process of lagging strand DNA replication.

A subject from a telomere possessing species can be any human or non-human subject. Non-human subjects encompass any suitable organism, including non-human mammals, vertebrate and invertebrate animals, plants, microorganisms, and the like. The described methods are directed to subjects from which quantification of critically short telomeres can provide clinical and research diagnostic information, such as for human and non-human veterinary subjects. However, the described methods are equally applicable for use in quantifying critically short telomeres in invertebrate and non-animal organisms, some of which, such as various fungi and protozoa, are used as model organisms.

In particular embodiments, the described methods are used to quantify critically short telomeres in a sample from a telomere possessing species. Such samples include, but are not limited to, samples isolated from a subject and from which DNA is isolated for use in the described methods. In other embodiments, such samples include tissue(s) or cells of a telomere possessing species, which are then cultured in vitro, and from which DNA is isolated. Any cellular or tissue sample with telomere-containing chromosomes could potentially be processed for use in the described methods. In particular embodiments, the subject of the described methods is a human or non-human mammalian subject. In other embodiments, the sample is a blood, buccal, or a suspected tumor biopsy sample. In some embodiments, the sample is newly obtained from the subject. In other embodiments, the sample is a stored sample. In some embodiments in which multiple samples are used to compare critically short telomeres over time, the samples can be a combination of sample(s) stored by standard methods in the art and newly isolated samples. In other embodiments, the samples have all been stored.

DNA for use in the described methods can be DNA that is isolated by any standard method for preparing chromosomal DNA from cellular material. It will be appreciated that in certain embodiments the chromosomal DNA is significantly removed (i.e., isolated) from its cellular source such that it is relatively pure. In other embodiments, the DNA is isolated from the cellular context only to that extent necessary to allow a user to perform the described method, such as to allow for restriction enzyme activity.

In the described methods, the sample DNA is digested by one or more restriction enzymes under standard conditions sufficient to allow for the restriction enzyme digest to proceed. Any one or combination of restriction enzymes can be used, but cannot cut the chromosome within the telomeric sequence, and preferably cuts close to the 5′ end of the telomeric region of the chromosome. Additionally, the restriction enzyme(s) used in the described methods must also produce a non-telomeric fragment no greater in length than the largest telomere-containing fragment to be analyzed, but large enough to be able to act as template for the described amplification reaction, and which contains the single copy non-telomeric sequence to be amplified and used in the described methods for quantifying critically short telomeres. In particular embodiments, the single copy non-telomeric sequence is found in the same restriction fragment as the telomeric sequence (i.e., the same DNA fragment). In other embodiments, the single copy non-telomeric sequence is found in a different fragment.

As the disclosed methods are applicable to any telomere possessing species, there is no requirement to employ a specific enzyme or combination of enzymes. Any one or combination of enzymes can be used, as long as the enzymes do not cut telomeric sequence, cut close to the 5′ end of the telomeric region of the chromosome, and produce restriction fragments of sufficient size to be compatible with the remainder of the methods as described.

A restriction enzyme(s) for use in the described methods is any enzyme or combination of enzymes that recognizes, targets, and cuts a specific DNA nucleotide sequence, leaving a blunt or a staggered (so called “sticky”) end. In particular embodiments, a restriction enzyme will cut the DNA at the target site. In other embodiments, it cuts the DNA outside of the recognized DNA sequence (e.g., Type-II and Type lis restriction enzymes). Restriction enzymes typically have recognition sequences that vary in nucleotide number from 4 (such as by the Msel enzyme) to 6 (by the EcoRI enzyme), to 8 (by the Notl enzyme). It will be appreciated that restriction enzymes that have shorter recognition sequences will therefore cut a given DNA sequence more frequently than an enzyme with a longer recognition sequence. Accordingly, digestion of a chromosome with a “short sequence cutter” will result in more restriction fragments than digestion with a “long sequence cutter.” Other factors in selecting the one or more restriction enzyme for use in the described methods include variation in activity dependent on epigenetic factors (e.g., methylation status) and variation in activity dependent on incubation conditions. Therefore, some restriction enzymes can be used simultaneously to digest DNA in a single reaction, whereas others must be used in sequence to maintain buffering conditions sufficient to allow for the specific digest to proceed. Information on selection and use of restriction enzymes for the current methods can also be obtained from enzyme suppliers such as New England Biolabs (Beverly, MA), Promega (Madison, WI), or Invitrogen/ThermoFisher (Carlsbad, CA).

Following restriction enzyme digestion, the resultant digested DNA (“restriction fragments”) are separated by size by any suitable method known to the art. In a particular embodiment, the restriction fragments are separated by gel electrophoresis, such as agarose, acrylamide, or capillary gel electrophoresis using standard methodology. In another embodiment, the restriction fragments are separated by standard chromatography methods. In a particular embodiment, separation as well as DNA fragment isolation are accomplished by way of an automated variation on gel electrophoresis, such as but not limited to the BluePippin automated system (Sage Science, Beverly MA). Methods of DNA fragment separation are well known in the art. While standard, such separation methods are selected and adapted for use in the current methods of quantifying critically short telomeres, and are selected and optimized to separate DNA fragments of particular sizes. For example, agarose gel electrophoreses typically uses gels of about 0.7% agarose for many standard applications. In a particular embodiment in which the critically short telomere sequences are found within a DNA fragment less than 500 bp in length, agarose gel electrophoresis can be used, but it will in some embodiments be optimized by using a gel with a higher percentage of agarose, such as 2% or 3%. In another example, in which the telomeric sequence is found within a larger restriction fragment, such as between 1.0-2.0 kb, or between 1.5-3.0 kb, the percentage of agarose can be less, such as 1%, 0.7%, and the like.

Isolation of DNA following fragment separation is achieved by standard methodologies. Any method of eluting DNA from a separation medium, such as but not limited to agarose or acrylamide gels, can be used. In particular embodiments, elution of DNA from an agarose gel fragment is accomplished by use of a kit from common suppliers such as, but not limited to Qiagen and Promega. In other embodiments, well-known electroelution and dialysis methods can be used to elute DNA from a gel fragment. In still other embodiments, automated systems can be used that combine electrophoresis, including agarose gel electrophoresis, and isolation of a given fragment size or range of sizes. For example, in one embodiment, such automated systems can isolate a 1.5 kb DNA fragment; in another embodiment, such systems can isolate a DNA fragment between 0.5 kb or 1.0 kb and 1.5 kb; and in yet another illustrative embodiment, such systems can isolate all DNA fragments in a sample (e.g. resultant from a restriction digest) that are up to 1.5 kb.

In particular embodiments of the described methods, restriction fragments are isolated that are for example between about 0.5 kb or 1.0 kb to about 1.5 kb in length. In another embodiment, restriction fragments of greater size, for example from about 1.5 kb to about 3.0 kb in length are isolated. Such fragments can be isolated by the described methods. While isolation of such fragments can remove some interstitial telomeric sequences (ITS) from the fragments to be amplified (which would dampen the “signal” of critically short telomeres being detected), the recently completed “telomere to telomere” sequence of the human genome indicates a greater number and size distribution of ITSs than previously appreciated (Nurk et al., Science 376:44-53, 2022). Accordingly, it is possible that not all ITSs are removed, even when critically short telomeres are detected in relatively larger fragments of 1.0 kb or 1.5 kb. It is appreciated however, that when performed on samples removed from the same subject over time, the presence of ITSs in the isolated DNA fragment has lesser impact on the precision of the described methods.

Amplification of (a) telomere and (b) single copy non-telomeric target sequences can be carried out by any method known to the art of DNA amplification. In particular embodiments, standard methods of PCR, including qPCR and ddPCR (see U.S. Pat. No. 9,347,094) can be used to amplify the indicated sequences.

According to standard methodologies, in particular embodiments, amplification of telomere sequences can be achieved by PCR using forward and reverse primers, for example wherein the forward primer hybridizes to non-telomeric sequences that are 5′ to the start of the telomeric sequence, and wherein the reverse primer hybridizes within the telomeric sequence. In another embodiment both forward and reverse primers hybridize within the telomeric sequence.

In particular embodiments, amplification of (a) telomeric and (b) non-telomeric single copy sequences can be performed in the same amplification reaction (i.e., within the same tube), and can in certain embodiments include use of a sequence-specific detection probe. In other embodiments, amplification of the telomeric sequence is carried out separately from amplification of non-telomeric single copy sequence (i.e. in different tubes).

In particular embodiments of the described methods, telomeric and non-telomeric amplification products are detected and quantitated as part of the amplification process (e.g., during qPCR, ddPCR, and the like). For example, amplification products are detected by a signal from a detectable label conjugated to at least one of the amplification primers used to amplify a target sequence. In other embodiments, amplification products are detected and quantitated by a similar detectable label and through use of standard electrophoresis methods, such as in a polyacrylamide gel. In such embodiments, loading controls are included for normalization purposes. Relative amounts of critically short telomeres are determined by dividing the quantitated value of the amplified telomeric sequence by the quantitated value of the amplified non-telomeric sequence.

In particular embodiments, the noted methods are carried out in parallel with a control sample which has a known relative amount of critically short telomeres, such as but not limited to a standard or a sample taken from the same subject, but at an earlier time.

In other particular embodiments, telomeres of average, rather than critically short telomeres are measured as a control or for comparison. Telomeres are determined and quantified by amplifying telomeric and non-telomeric sequences directly from chromosomal DNA, which has not been digested and separated.

### III. Kits

Additionally described herein are kits for carrying out the described methods for identifying and quantifying critically short telomeres. The contents of such kits will depend on the telomere possessing species, which is being assayed, and accordingly the chromosome that is being examined. However, all kits will include at least the necessary restriction enzyme or enzymes, reagents, buffers, and primers, and optionally probes necessary to digest the target telomere-containing DNA, amplify the target telomeric and non-telomeric sequences, and optionally probe for the target telomeric and non-telomeric sequences. In particular embodiments, the kits also include additional reagents necessary for PCR amplification, including buffers, nucleotides, and thermal stable DNA polymerase (e.g., Taq, Pfu, and the like, including engineered versions thereof). In other embodiments, the kits include precast agarose gels and reagents necessary to elute DNA from the gel. In still other embodiments, kits include control DNA with a known relative quantity of critically short telomeres.

In all embodiments of the described kits, instructions are provided for using the provided components to detect and quantify critically short telomeres. Instructions can be provided in any medium known to the art, including but not limited to paper instructions and on digital medium. Instructions can also be provided and accessed by an URL and/or a scannable code provided with the kit, such as a standard QR code.

### IV. Applications for Methods of Detecting and Quantifying Critically Short Telomeres

An increased proportion of critically short telomeres in a cell or organism is understood to be strongly associated not only with cellular senescence and natural aging processes, but also with a range of cellular and organismal pathologies (Mangaonkar and Patnak, Mayo Clin. Proc., 93:904-916, 2018). Accordingly, the methods described herein for detecting and quantifying critically short telomeres can be used in a variety of applications from diagnostic and prognostic methods to therapeutic compound screening.

In a particular embodiment, the described methods can be used to determine the csTelomere score of a subject. In such embodiments, a test sample, such as a blood or buccal sample, is obtained from a subject and processed as described to determine the relative quantity and proportion of critically short telomeres. The relative value obtained for the subject is then compared to one or more control values from subjects of known csTelomere score. In a particular embodiment, the control value is an historic control value of a subject of young, average, and or/increased csTelomere score (i.e., having a higher proportion of critically short telomeres). In another embodiment, the control value is experimentally obtained concurrently with the relative value from the test subject. In particular embodiments, the control value is a csTelomere score determined from a sample taken from the subject at an earlier time point, in order to establish a baseline of comparison for critically short telomeres at any future time point. In further embodiments, the determination that a subject is of abnormal csTelomere score can indicate that the subject should be further evaluated and/or treated to slow or reverse the increase in critically short telomeres and associated abnormal senescence and aging. Conversely, the determination that a subject has a lower csTelomere score in comparison to a baseline taken from the same subject at an earlier time point can indicate efficacy of a given treatment.

In a related embodiment, the described methods can be used to monitor the efficacy of a therapy for a disease or condition associated with critically short telomeres.

In a particular embodiment, the condition can be the normal course of aging. A subject of congruent chronological and csTelomere score can be provided with treatments intended to decrease the rapidity of the aging process or reverse certain biological consequences of the aging process (e.g., decreased system function such as immune or pulmonary systems). The efficacy of such longevity enhancing treatments can be determined by comparing a determined pre-treatment value of critically short telomeres in the subject with measurement of critically short telomeres in the subject during or following a prescribed course of treatment.

In another embodiment, the disease or condition can be a Short Telomere Syndrome (see Mangaonkar, 2018), such as diseases associated with an organ comprised of cells that frequently divide including bone marrow, liver, lungs, and the immune system. In such embodiments a starting value for the proportion of critically short telomeres is determined. The determination that a subject who presents with certain physical pathologies associated with shorter than average, and particularly critically short, telomeres can be indicative that the subject is in need of a treatment directed at curing a telomere disfunction in place of or in addition to treatments aimed at a particular pathological phenotype. The efficacy of such treatments, particularly those aimed at curing telomere disfunction, can be determined by assaying for critically short telomeres by the methods described herein and comparing the determined value to that determined before treatment began. In a particular embodiment, such assays determine that the treatments are effective and indicate that it could be of benefit to continue a given treatment. In another embodiment, such assays determine that the treatments are ineffective, thereby indicating that a different treatment should be administered.

In a related embodiment, the described methods can be used to monitor the efficacy of a therapy for a disease or condition associated with abnormally long telomeres, such as certain diseases associated with aberrant cellular proliferation. Such methods proceed similarly to those described above, except that in particular embodiments, it is therapeutically beneficial to increase the proportion of critically short telomeres in a given cell or tissue, thereby driving the cells towards increased senescence and halting the aberrant proliferation.

In a particular embodiment, conditions and diseases related to critically short telomeres and abnormal csTelomere score are resultant from inactive telomerase in a subject. Accordingly, in particular embodiments, the described methods can be used as described above to determine the efficacy of treatments that specifically target telomerase activity, and/or particularly provide active agents or treatments that provide telomerase, increase telomerase activity, and/or activate telomerase. Particular non-limiting examples of such agents or treatments include activation of endogenous or exogenous telomerase expression or provision of exogenous telomerase, including by compounds such as TA-65 and influenced by lifestyle changes such as diet and exercise. Other related treatments that can be prescribed to affect cs telomeres include treatments that inhibit cellular senescence in general, such as, but not limited to senolytic treatments, hyperbaric oxygen treatments, and the like. In other embodiments measurement of cs telomeres can indicate a need for cell replacement therapy, such as use of pluripotent stem cells, including induced pluripotent stem cells. Measurement of cs telomeres in a given tissue from a subject can also in certain embodiments detect the successful engraftment of stem cells and/or indicate a need for additional treatments, including additional cell treatments.

In a further particular embodiment, the described methods can provide an assay tool for screening compounds that effect telomere length. In a particular embodiment, cells or tissues in culture with a known or experimentally determined relative value of critically short telomeres are contacted with a test agent and incubated for a time sufficient for the test agent to have a biological effect. Following the incubation period, the described methods are used to determine the relative amount and proportion of critically short telomeres in the cultured cells or tissue. The efficacy of the test agent will depend on the given goal. In particular embodiments, the screen can be for an agent that promotes increased telomere length, thereby decreasing the relative amount and proportion of critically short telomeres. In other embodiments, the screen can be for an agent that inhibits telomere length, thereby increasing the relative amount and proportion of critically short telomeres. It will be appreciated that the described screening methods can be adapted for high-throughput screens of active agents.

In a further embodiment, the described methods can be used to assay quality control or viability of immortalized cell lines. Maintenance of average telomere length is a characteristic of certain cultured cells, such as but not limited to immortalized cell lines for research or stem cells intended for research and/or clinical applications. The viability of such cultures can be determined by measuring the amount and proportion of critically short telomeres as described herein. For such methods, a baseline of critically short telomeres can be determined for a given cell line in storage, immediately upon plating, or following a first passage. Once a baseline is determined, it can be used as a control for further comparison of newly determined values of critically short telomeres of the cells in storage after a given number of months or in culture after a given number of passages. A marked increase in critically short telomeres as determined by such methods can indicate the decreased viability of the cells in culture or in storage. An advantage of the described methods over measurement of average telomere length is that critically short telomeres are associated with cellular senescence, and thus provide a more reliable characteristic by which to determine the viability of a cell line.

The following examples are provided to illustrate certain particular features and/or embodiments. These examples should not be construed to limit the disclosure to the particular features or embodiments described.

### EXAMPLES

### Example 1: Quantitation of Critically Short Telomeres

This example describes and demonstrates a method for quantitating critically short telomeres.

To detect and quantitate critically short telomeres in a sample, it is necessary to first establish reliability of the amplification, detection, and quantitation of a known amount of critically short telomeres in a sample of human genomic DNA. To that end, different amounts of a 900 bp telomere fragment, corresponding to a critically short telomere size, were added to a human genomic DNA sample. Critically short telomere amount was measured as follows.

DNA was digested with a combination of four restriction endonucleases, CviAII, NdeI, BfaI, MseI, according to manufacturer's instructions. The resultant digested DNA was separated on a 0.75% agarose gel. Gel sections between 0.5 and 1.5 kb DNA fragments were excised, and DNA was eluted from the sections.

A telomere-specific sequence (Tel) was PCR amplified with telomere-specific forward (5′—ACACTAAGGTTTGGGTTTGGGTTTGGGTTTGGGTTAGTGT—3′(SEQ ID NO: 1)) and reverse (5′—TGTTAGGTATCCCTATCCCTATCCCTATCCCTATCCCTAACA—3′(SEQ ID NO: 2)) primers. Simultaneously, a single-copy non-telomeric sequence (Non-tel) found in a post-digestion DNA fragment between 0.5 and 1.5 kb was PCR amplified with the following forward (5′—CTAAGCTGACAGCCGGGTAG—3′(SEQ ID NO: 3)) and reverse (5′—TGTGGTAGTTCGCCCCATTC—3′(SEQ ID NO: 4)) primers. Multiplex PCR was carried out using the Biorad QX200 Droplet digital PCR system. The presence of amplified telomeric sequence was probed by: TAACCCAAACCCAAACCCAAACCCAAA (attached to 6-FAM color; SEQ ID NO: 5). For detection of critically short telomeres in larger isolated fragments between 1.5 and 3.0 kb, a single copy genomic sequence of 1563 bp was PCR amplified with the following forward (5′—ACGGAAACCTTGGAGCAGAG—3′(SEQ ID NO: 6)) and reverse (5′—GCCACGTTCATTGCACAGTT—3′(SEQ ID NO: 7)) primers. The presence of the amplified single copy sequence was probed by GGAGGAATACGGAGGCGGGGA (attached to HEX color; SEQ ID NO: 8).

The level of critically short telomeres in each sample was determined by dividing the signal received at the telomere reaction (Tel), by the signal received at the single copy reaction (Non-tel).

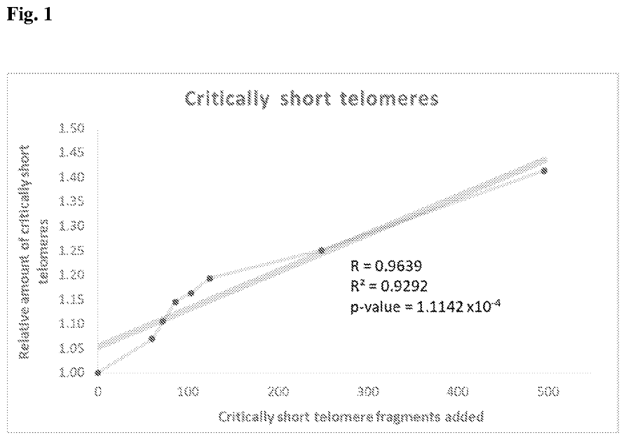

As shown in FIG. 1, the described method for measuring critically short telomeres shows high linearity for measuring the critically short telomeres of 0.5-1.5 kb in length that were added to a mixed sample of human genomic DNA. Accordingly, the described method can accurately quantify critically short telomeric “signal” in the context of genomic “noise”.

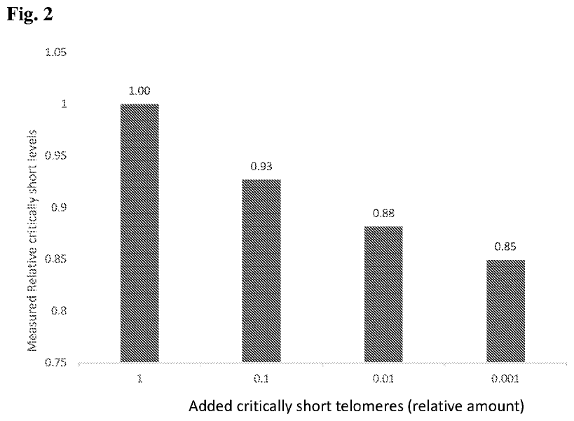

The described method was repeated to detect the relative amount of critically short telomeres from a subject of older biological age that was added to a genomic sample of a subject of younger biological age. Briefly, a genomic DNA sample of an older individual was digested using the restriction enzymes as above, and then extracted in the range between 1.5-3.0 kb (using a BluePippin device). Different amounts from this “critically short sample” were added to an undigested genomic DNA of a different individual, and these samples were treated as starting material for the method as described. As shown in FIG. 2, the described method detects the decreased amount of added cs telomeres in the expected trend, though with decreased specificity in view of the “noise” of the starting genomic sample which itself includes cs telomeres.

### Example 2: Quantitation of Critically Short Telomeres in Differently-Aged Subjects

This example shows the quantitation of critically short telomeres in several differently-aged human subjects, and detects an increased critically short telomere score in some subjects of increased age.

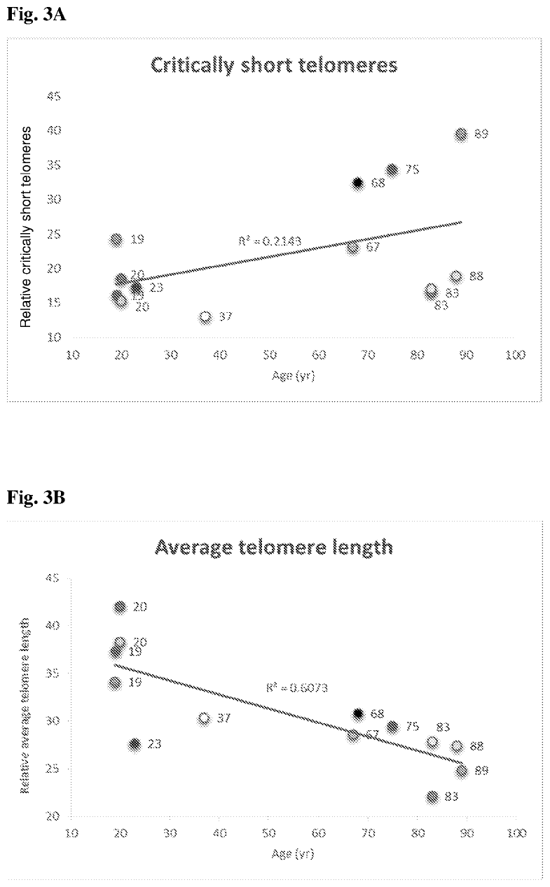

Using the method described in Example 1, telomere length measurements were determined from whole blood samples taken from 13 people of ages 19 to 89 years old. The results are presented in FIGS. 3A and 3B, which show measurement of critically short telomeres between 0.5 and 1.5 kb in length (FIG. 3A). For comparison, average telomere length for intact DNA was measured by ddPCR using the same primers and probes as described, but without digestion and separation of DNA fragments (comparison of average telomere length is presented in FIG. 3B). As shown in the figures, while average telomere length tends to get shorter with chronological age, a different pattern is observed while measuring critically short telomeres. When comparing the older group (above 60 y/o) with the younger group (below 40 y/o) the values of critically short telomeres for individuals in the older group can be as low as for the younger group, or much higher.

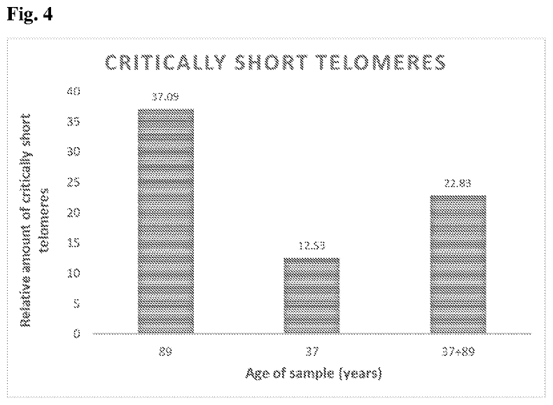

As a further control for the method, a 1:1 Mix of DNA samples from two people with different levels of critically short telomeres were measured as described and shown to produce the corrected average (FIG. 4). Critically short telomeres between 0.5 and 1.5 kb in length were measured as described above for DNA isolated from people aged 89 and 37 years old, and also for a 1:1 mix of these two DNA samples (“37+89”). This assay shows that a 1:1 Mixing of the DNA from a 89 year old (which has a high level of critically short telomeres) and the DNA from a 37 year old (which has a low level of critically short telomeres) produce a result very close to the mathematical average of the two original samples, further demonstrating the accuracy of the described method.

### Example 3: Detection of Critically Short Telomeres Over Time

This example shows the quantitation of critically short telomeres in foreskin fibroblast cells at a different passages, and detects an increased critically short telomere score in cells that have undergone more divisions (late passage).

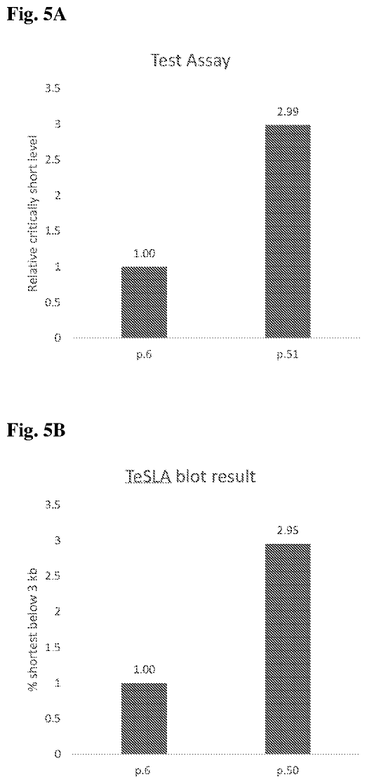

Using the method described in Example 1, telomere length measurements were determined from the same foreskin fibroblast cell line in the early passage (p.6) and late passage (p.51). The results are presented in FIGS. 5A and 5B, which show measurement of the relative critically short telomeres of 1.5-3.0 kb in length. As shown in the figures, the critically short telomeres increase in the late passage. As shown in the comparison of FIGS. 5A and 5B, the result using the method described herein is the same as that obtained assaying for the percent of shortest telomere below 3 kb using the TeSLA method (Lai et al., Nat. Comm. 8:1356, 2017).

These results demonstrate that the methods described herein can be used to monitor the change in critically short telomeres between samples obtained from a subject over a given period of time.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for quantifying critically short telomeres, starting from a DNA sample, in a subject from a telomere-possessing species or sample therefrom, the method comprising:
digesting DNA from the subject or sample therefrom with at least one restriction enzyme, wherein the at least one restriction enzyme cuts proximal to, but does not cut within, a telomeric sequence;
separating the digested DNA by size;
isolating from the separated DNA, polynucleotide fragments shorter in length than an average telomere length for the sample;
amplifying from the isolated polynucleotide fragments: (a) a telomeric-specific sequence; and (b) a non-telomeric single copy sequence;
quantitating the products of (a) and (b); and
determining an amount of the critically short telomeres by dividing the quantitated amount of (a) by the quantitated amount of (b).

2. The method of claim 1, wherein the DNA is digested with multiple restriction enzymes.

3. The method of claim 1, wherein the digested DNA is separated by gel electrophoresis.

4. The method of claim 3, wherein the polynucleotide fragments are isolated from the gel.

5. The method of claim 3, wherein the polynucleotide fragments are isolated from the gel by removing a gel section containing the fragments, and eluting the DNA from the gel section.

6. The method of claim 1, wherein the isolated polynucleotide fragments are no larger than about 1.5 kilobases or about 3.0 kilobases in length.

7. The method of claim 1, wherein the isolated polynucleotide fragments are between about 0.5 kilobases and about 1.5 kilobases in length, between about 1.0 kilobases and about 1.5 kilobases, or between about 1.5 kilobases and about 3.0 kilobases in length.

8. The method of claim 1, wherein (a) and (b) are amplified in the same PCR reaction.

9. The method of claim 1, wherein amplification and quantitation of (a) and (b) is by quantitative PCR.

10. The method of claim 9, wherein the quantitative PCR is real time PCR or digital PCR.

11. (canceled)

12. (canceled)

13. The method of claim 1, wherein the sample is a blood or buccal sample.

14. A kit for carrying out the method of claim 1.

15. A method for quantifying over time critically short telomeres, starting from a DNA sample, in a subject from a telomere-possessing species or sample therefrom, the method comprising:
a) digesting DNA from the subject or sample therefrom with at least one restriction enzyme, wherein the at least one restriction enzyme cuts proximal to, but does not cut within, a telomeric sequence;
b) separating the digested DNA by size;
c) isolating from the separated DNA, polynucleotide fragments shorter in length than an average telomere length for the sample;
d) amplifying from the isolated polynucleotide fragments: (i) a telomeric-specific sequence; and (ii) a non-telomeric single copy sequence;
quantitating the products of (i) and (ii);
e) determining an amount of the critically short telomeres by dividing the quantitated amount of (i) by the quantitated amount of (ii);
f) repeating steps a) to e), wherein the DNA is obtained from the subject at a later time point in comparison to the DNA used in steps a) to e); and
g) comparing the amount of the critically short telomeres determined in step f) with the amount of the critically short telomeres determined in step e).

16. The method of claim 15, wherein the later time point is hours, days, months, or years.

17. The method of claim 15, wherein step f) is repeated at one or more additional later time points.

18. The method of claim 15, wherein the subject is undergoing a treatment for a disease or condition associated with increased critically short telomeres, and wherein a decrease in the amount of the critically short telomeres over time indicates efficacy of treatments for the disease or condition.

19. The method of claim 15, wherein the subject is undergoing a treatment for a disease or condition associated with decreased critically short telomeres, and wherein an increase in the amount of critically short telomeres over time indicates efficacy of treatments for the disease or condition.

20. The method of claim 19, wherein the disease is a cancer, and wherein the therapy is chemotherapy.

21. The method of claim 15, wherein the isolated polynucleotide fragments are no larger than about 1.5 kilobases or about 3.0 kilobases in length.

22. The method of claim 15, wherein the isolated polynucleotide fragments are between about 0.5 kilobase and about 1.5 kilobases in length, between about 1.0 kilobases and about 1.5 kilobase, or between about 1.5 kilobases and about 3.0 kilobases in length.


SEQUENCE LISTING (8 sequences)

SEQ ID NO: 1 - DNA, 40 bp, Artificial Sequence
  Synthetic oligonucleotide
acactaaggtttgggtttgggtttgggtttgggttagtgt

SEQ ID NO: 2 - DNA, 42 bp, Artificial Sequence
  Synthetic oligonucleotide
tgttaggtatccctatccctatccctatccctatccctaaca

SEQ ID NO: 3 - DNA, 20 bp, Artificial Sequence
  Synthetic oligonucleotide
ctaagctgacagccgggtag

SEQ ID NO: 4 - DNA, 20 bp, Artificial Sequence
  Synthetic oligonucleotide
tgtggtagttcgccccattc

SEQ ID NO: 5 - DNA, 27 bp, Artificial Sequence
  Synthetic oligonucleotide
taacccaaacccaaacccaaacccaaa

SEQ ID NO: 6 - DNA, 20 bp, Artificial Sequence
  Synthetic oligonucleotide
acggaaaccttggagcagag

SEQ ID NO: 7 - DNA, 20 bp, Artificial Sequence
  Synthetic oligonucleotide
gccacgttcattgcacagtt

SEQ ID NO: 8 - DNA, 21 bp, Artificial Sequence
  Synthetic oligonucleotide
ggaggaatacggaggcgggga